(19) United States
(12) Patent Application Publication
(10) Pub. No.: US 2003/0232021 A1
(43) Pub. Date: Dec. 18, 2003

(54) Method for the treatment of dental caries caused by streptococci mutans infection by inhibiting energy storage and utilization

(75) Inventor: Alan M. Schechter, Long Beach, CA (US)

Correspondence Address: BIRCH STEWART KOLASCH & BIRCH, PO BOX 747, FALLS CHURCH, VA, 22040-0747, US

(73) Assignee: Pyro Pharmaceuticals, Inc., Long Beach, CA

(21) Appl. No.: 10/411,706

(22) Filed: Apr. 11, 2003

Related U.S. Application Data
(60) Provisional application No. 60/372,307, filed on Apr. 12, 2002.

(51) Int. Cl. A61K 31/7076; A61K 7/16

(52) U.S. Cl. 424/49; 514/47

(57) ABSTRACT

A method and pharmaceutical composition for inhibiting infections of S. mutans by the addition of said composition to toothpaste or mouthwash, by inhibiting the production of ADP-glucose, particularly by inhibiting the activity of ADP-glucose pyrophosphorylase or glycogen synthase.

### CROSS-REFERENCE TO RELATED APPLICATION

This application claims priority on provisional Application No. 60/372,307 filed on Apr. 12, 2002, the entire contents of which are hereby incorporated by reference.

### FIELD OF THE INVENTION

The present invention is directed to the use of bacterial enzymes as targets for antibiotic therapy and the treatment of dental caries caused by S. mutans infection, particularly by inhibiting enzymes involved in energy storage and utilization.

### BACKGROUND OF THE INVENTION

Starch, a complex polymer of glucose, is present in most green plants in practically every type of tissue and is the major intracellular reserve polysaccharide in photosynthetic organisms. The glucan accumulates during development of storage or seed tissues and is catabolized to serve as a source of energy. In the animal kingdom, as well as in fungi, yeast and bacteria, the primary reserve polysaccharide is glycogen. Glycogen is a polysaccharide containing linear molecules with α-1,4 glucosyl linkages and is branched via α-1,6-glucosyl linkages. Although glycogen is analogous to starch with regard to linkages, glycogen exhibits a different chain length and a different degree of polymerization.

The following common reactions are shared by the biosynthetic pathways of bacterial glycogen and of starch in algae and higher plants:

(1) ATP+α-Glc-1-P [symbol] ADPlc+PPi

(2) ADPGlc+α-1,4-glucan→ADP+α-1,4-glucosyl-α-1,4-glucan

(3) Elongated α-1,4-glucan chain→Branched α-1,6-α-1,4-glucan

In step (1), ADP Glucose (ADPGlc) is synthesized from ATP and glucose-1-phosphate in the rate-limiting reaction which is catalyzed (in plants and bacteria) by ADP-glucose

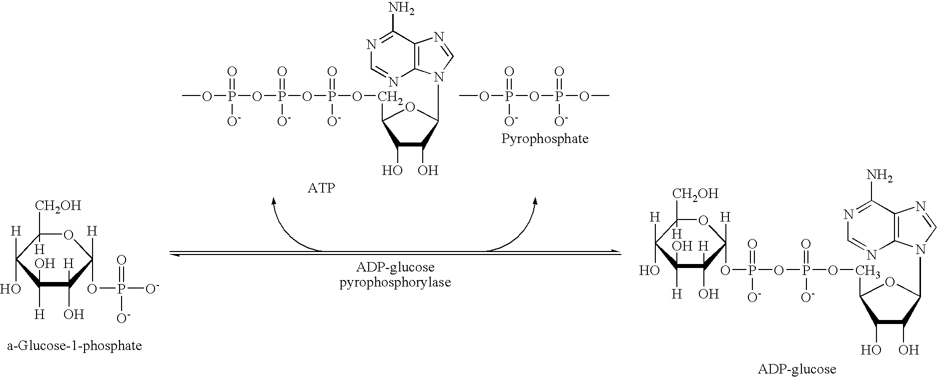

pyrophosphorylase (also referred to as ADPGlc PPase; or ADPG PPase, or glucose-1-P adenyltransferase, or as enzyme EC 2.7.7.27). The chain elongation step (2) is catalyzed by glycogen synthase (also referred to a GS, gly A, or as enzyme EC 2.4.1.21).

1. ADP-glucose phyrophosphorylase

The reaction scheme catalyzed by ADPGlc PPase is shown below: Significant research has led to cloning and sequencing genes which code for ADP-glucose pyrophosphorylase in plants for the purpose of modulating sucrose and starch content in plants. For example, U.S. Pat. Nos. 5,498,831 and 5,773,693 to Burgess et al. described the sequence of Pea ADP-glucose pyrophosphorylase subunit genes and the use of those genes to transform plant cells in order to provide plants that have increased sucrose content.

U.S. Pat. No. 6,184,438 to Hannah describes mutant genes encoding plant ADP-glucose pyrophosphorylase and the use of those genes to produce transformed plants having enhance germination characteristics but without any diminishment in food quality or flavor.

U.S. Pat. No. 6,057,493 to Willmitzer et al. describes the use of anti-sense DNA sequences encoding ADP-glucose pyrophosphorylase from potato to produce transformed plants with a reduction in starch concentration and an increase in the concentration of at least sucrose.

The genes which code for several bacterial ADPGlc PPases have also been cloned and recombinant enzymes have been prepared. See for example, Preiss, J., and M. N. Sivak, M. N. (1998) Genet. Eng. N.Y.) 20, 177-223;Preiss, J. (1996) In Escherichia coli and Salmonella typhimurium: Cellular and Molecular Biology (Neidhart, F. C., Ed.), 2^{nd} ed., Vol 1 pgs. 10115-1024, ASM Press; Preiss, J., and Romeo, T. (1989) Advances in Microbial Physiology 30, 183-238. DNA sequences have also been elucidated for certain bacterial ADP-glucose pyrophosphorylase enzymes. For example, U.S. Pat. No. 5,349,123 to Shewmaker et al. describes a nucleic acid construct which encodes an E. coli ADP-glucose pyrophosphorylase and the use of that construct to transform plant cells to modify biosynthesis of a glucan in the plant.

2. Glycogen Synthase

The reaction scheme in bacteria catalyzed by glycogen synthase is shown below:

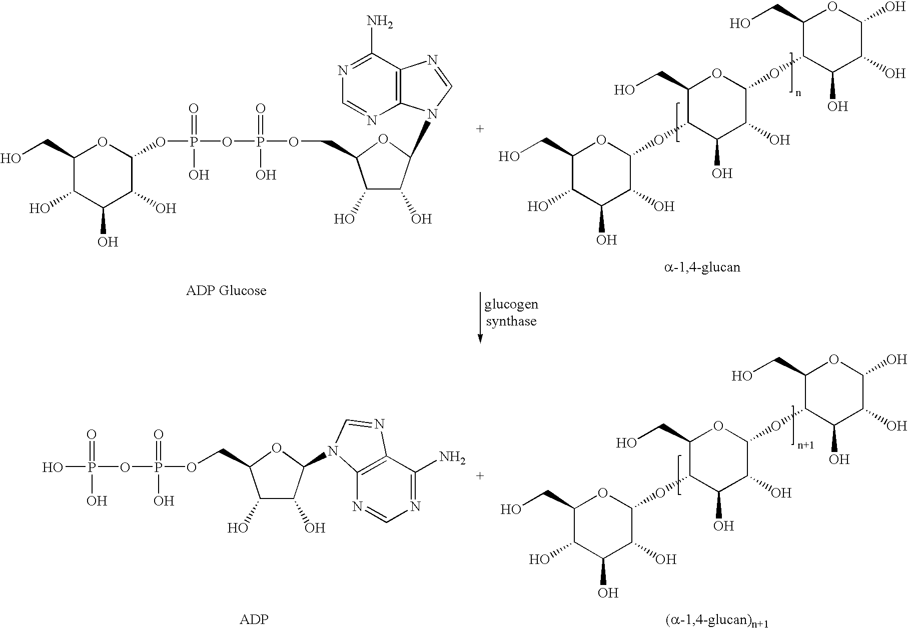

The “corresponding” reaction in mammals is similar except that the nucleoside diphosphate sugar substrate is UDP glucose, and the catalyzing enzyme is EC 2.4.1.11.

Genes encoding mammalian glycogen synthases have been cloned and sequenced (See Browner et al., Proc. Nat. Acad. Sci. (1989) 86:1443-1447;Bai et al., J. Biol. Chem. (1990) 265:7843-7848), and the genes encoding bacterial glycogen synthases have also been cloned and sequenced. (See for example U.S. Pat. No. 5,969,214.)

Dental caries is the progressive loss of tooth mineral, followed by bacterial invasion into the demineralized tooth. It is a relatively complex disease. The nature of caries can be described in terms of five interrelated factors. In addition to helping explain the nature of the disease, each factor gives guidance to how to prevent it and to how it can be cured.

Factor 1. Caries is a Bacterial Disease

There is abundant evidence that the initiation of caries requires a relatively high proportion of mutans streptococci within dental plaque. These bacteria adhere well to the tooth surface, produce higher amounts of acid from sugars than other bacterial types, can survive better than other bacteria in an acid environment, and produce extracellular polysaccharides from sucrose. When the proportion of S. mutans in plaque is high (in the range 2-10%) a patient is at high risk for caries. When the proportion is low (less than 0.1%) the patient is at low risk. Infection with S. mutans usually happens early in childhood by transmission from the mouths of parents or playmates. Because they are more acid tolerant than other bacteria, acid conditions within plaque favor the survival and reproduction of S. mutans. Two other types of bacteria are also associated with the progression of caries through dentin. These are several species of Lactobacillus lactis, and Actinomyces viscosus. These bacteria are also highly acidogenic and survive well in acid conditions.

Factor 2. Caries is Dependent on Dietary Sucrose

Dietary sucrose changes both the thickness and the chemical nature of plaque. S. mutans and some other plaque bacteria use the monosaccharide components (glucose and fructose) and the energy of the disaccharide bond of sucrose to assemble extracellular polysaccharides. These increase the thickness of plaque substantially, and also change the chemical nature of its extracellular space from liquid to gel. The gel limits movement of some ions. Thick gel-plaque allows the development of an acid environment against the tooth surface, protected from salivary buffering. Plaque which has not had contact with sucrose is both thinner and better buffered. A diet with a high proportion of sucrose therefore increases caries risk. Thicker plaque occurs in pits and fissures, just beneath the contact area and, in patients with poor oral hygiene, near the gingival margin.

Factor 3. Caries is Driven by Frequency of Eating

Each time that plaque bacteria come into contact with food or drink containing simple sugars (monosaccharides such as glucose and fructose, and disaccharides such as sucrose, lactose and maltose) they use them for their metabolic needs, making organic acids as a metabolic by-product. If these acids are not buffered by saliva they dissolve the surface of the apatite crystals of adjacent tooth structure. This is called demineralization. In thick gel-plaque the pH falls within seconds of contact with dietary sugars, and it can stay low for up to 2 hours. When the pH is neutral the same crystals can re-grow, using calcium, phosphate and fluoride from saliva. This is called remineralization. Caries begins and progresses when demineralization outweighs remineralization. Caries therefore depends on the balance between demineralization and remineralization, i.e. on the frequency of eating (and on the microbial composition of the plaque and its chemical nature and thickness, on the local fluoride concentration and on the buffering capacity of saliva). A frequent pattern of eating therefore increases caries risk.

Factor 4. Caries is Modified by Fluoride

The mineral of enamel, cementum and dentin is a highly-substituted calcium phosphate salt called apatite. The apatite of newly-formed teeth is rich in carbonate, has relatively little fluoride and is relatively soluble. Cycles of partial demineralization and then remineralization in a fluoride-rich environment creates apatite which has less carbonate, more fluoride and is less soluble. Fluoride-rich, low carbonate apatite can be up to ten times less soluble than apatite low in fluoride and high in carbonate. Topical fluoride also inhibits acid production by plaque bacteria. Fluoride in food and drinks, fluoride in dentifrices and oral rinses and gels, and fluoride in filling materials can therefore all reduce the solubility of teeth, helping to reduce caries risk. These effects are very beneficial, but the amounts of fluoride which can be added to the diet or used topically are limited by safety considerations. High levels of dietary fluoride can cause mottling of tooth enamel during tooth formation, while swallowing even higher levels can cause symptoms of poisoning.

Factor 5. Caries is Modified by Saliva

High flow-rate saliva is a very effective buffer. The balance between demineralization and remineralization can therefore be altered substantially by the rate of salivary flow. Flow is decreased by salivary gland pathology (as occurs in several connective tissue disease and which can follow radiotherapy and cancer chemotherapy), by many mood-altering drugs and some drugs used in other medical treatment, in dehydration and during sleep. Flow increases naturally during vigorous chewing. A maximum salivary flow rate (which can be tested by collecting all saliva which chewing wax or gum) of less than 0.7 mL/min. is associated with high caries risk.

How the Disease is Treated Today

The present, molecular concept of the nature of caries leads us to very different concepts of management of the disease. This, coupled with the widespread use of fluoride and the development of restorative materials which adhere to tooth structure and which (in some cases) do not leak, has revolutionized the prevention and cure of caries, as well as the repair of carious defects in teeth. The key features of the new care paradigms are summarized below.

Diagnosis

Since we understand caries to be a dynamic process which occurs at the molecular level we can diagnose the disease before irreversible loss of tooth structure occurs. It is now reasonable to state, on the basis of diagnosis, that some people do have the disease, while others do not. Detection of lesions at the macroscopic level can no longer be considered to be diagnosis, for two reasons—(1) the disease is present before lesions can be detected macroscopically and (2) large lesions remain after the disease is cured. Determination of risk state is a reasonable diagnostic goal, as is activity state.

Management

Treatment and Cure

The goal of treatment is now to change the local biochemistry so that the patient is no longer losing tooth mineral so that the disease is then cured and the patient healed. This is logical, ethical, appropriate and achievable. Caries can be treated by one or more of the following:

Changing the microflora, using agents such as topical chlorhexidine and topical fluoride

Reducing the amount of dietary sucrose, by dietary choice

Decreasing the frequency of eating, by dietary choice

Adding fluoride, particularly through daily application during tooth brushing

Increasing salivary flow, using mechanical stimulation during vigorous chewing to enhance flow, by changing drugs which reduce flow, or by using drugs to enhance flow

Cure is achieved when diagnostic tests show that the disease is no longer active and the risk is low.

Important characteristics for chemical antimicrobial substances directed against dental caries are their capability of inhibiting bacterial colonization-adhesion and their capacity to affect plaque growth-metabolic activity. They should also fulfill other demands such as, not interfering in other biological processes, being harmless towards the mucosa, and have a low toxicity, since parts of the substance may be swallowed. There are several antimicrobial substances that can be used in the oral cavity and with varying efficacy. “Varying efficacy” depends on many factors like vehicle, concentration, substantivity, duration of treatment and the co-operation of the patient.

Cations

1.1 Substance, Vehicles and Concentrations

Inter alia chlorhexidine, which has been known and used for about 30 years in Europe. Examples of other products that contain cations are metal ions (Cu²⁺, Zn²⁺ and Sn²⁺), alexidine (like chlorhexidine a bisbiguanide) cetyl pyridinium chloride (a quaternary ammonium compound), hexetidine (a synthetic hexahydropyridine) and sanguinaria extracts (a natural herbal extract).

1.2 Operation

Cations are attracted to the bacterial cell walls because of the substances' positive charge and the negative charge of the bacterial cell wall. Gram positive bacteria are more sensitive to cations since they are more negatively charged. S. mutans are Gram positive bacteria and are therefore very sensitive to cations.

Chlorhexidine, is both hydrophilic (charged) and hydrophobic (non-charged). Because of these characteristics, chlorhexidine can affect the cariogenic S. mutans. Which way of action that will dominate is also dependent on the concentration of the substance. Three general effects are seen:

Disturbing the normal membrane functions of the bacteria and especially mutans streptococci.

Interfering with the bacterial adhesion on the tooth or in the pellicle by affecting a surface enzyme.

Interfering with a glycolytic enzyme which leads to a reduced acid production by the bacteria.

Metal ions exhibit an additional way of action on bacteria. Two enzymes in the glycolysis may be disturbed. This leads to a reduced acid production by the bacteria.

1.3 Efficacy

The most frequently used substance in the cation group from a professional point of view is without any doubt chlorhexidine. Since it is the substance that best fulfill the demands of disturbing the membrane functions, being plaque-reducing and inhibiting the metabolism, chlorhexidine is also being used as a reference when other cationic agents are compared concerning their efficacy.

The duration of effect depends on the concentration and substantivity of the agent but also on the selection of vehicle. Below are a few examples of results: A study, performed in Bangkok, evaluated the possibility of reducing fissure caries development by using a chlorhexidine varnish in a split mouth method. “The results showed that: Cervitec varnish reduced fissure caries development significantly; the levels of salivary mutans streptococci at balseline were significantly correlated with caries status at baseline and with total caries increment over the two-year period; caries development in a fissure was significantly correlated to the level of plaque mutans streptococci at that same site; three months after the last varnish application, a certain reduction of mutans streptococci in plaque could be seen in the test teeth; comparing the size of the lesions, more large cavities were found in the untreated teeth”. Bratthall D, Serinirach R, Rapisuwon S, Kuratana M, Luangjarmekorn V, Luksila K, Chaipanich P. A study into the prevention of fissure caries using an antimicrobial varnish. Int Dent J 1995; 45: 245-54.

Mouthrinses containing hexetidine have inferior effects compared to those of chlorhexidine. This counts for cetyl pyridinium chloride and alexidine mouthrinses as well. This was seen in a single rinse test measured in 10 subjects. “Return to pre-rinse levels was seen for hexetidine after 90 min, cetyl pyridinium chloride after 3 hours, alexidine after 5 hours and chlorhexidine gluconate after 7 hours.” Roberts W R, Addy M. Comparison of the in vivo and in vitro antibacterial properties of antiseptic mouthrinses containing chlorhexidine, alexidine, cetyl pyridinium chloride and hexetidine. Relevance to mode of action. J Clin Periodontol 1981; 8: 295-310.

Hexetidine containing mouthrinses with increased concentrations from 0.10 to 0.14 per cent reaches equal efficacy as chlorhexidine 0.20 per cent mouthrinses may lead to desquamative lesions as a side effect. Based on “Scheie A. Modes of Action of Currently known Chemical antiplaque Agents other than Chlorhexidine. J Dent Res 68 (Spec Iss) 1989; 1909-1616. An in vitro study of SnF₂ gels demonstrate the effect of inhibiting among others mutans streptococci growth in plaque. “SnF₂ at a concentration of 0.4 per cent had a similar antibacterial effect to 0.12 per cent chlorhexidine. “Tseng C C, Wolf L F, Aeppli D M. Effect of gels containing stannous fluoride on oral bacteria—an in vitro study. Aust Dent J 1992; 37: 368-373.

“Sanguinarine containing mouthwashes and toothpastes appear to be less effective than chlorhexidine (Corsodyl).” Grenby T H. The use of sanguinarine in mouthwashes and toothpaste compared with some other antimicrobial agents. Br Dent J 1995; 178: 254-258. The substantivity is rather equal for a stannous fluoride containing toothpaste compared to chlorhexidine containing mouthrinse. The substantivity most likely reflect the plaque inhibitory properties. Based on “Elworthy A, Greenman J, Doherty F M, Newcombe R G, Addy M. The substantivity of a number of oral hygiene products determined by the duration of effects on salivary bacteria. J Periodontol 1996; 67: 572-576.

“On various antimicrobial agents and methods tested, the persistent reduction of mutants streptococci has been achieved by chlorhexidine varnishes, followed by gels and mouthwashes. The best clinical effect resulting in a considerable caries reduction has been obtained when persons highly colonized with mutans streptococci have been treated with gels and when the results of the antimicrobial measures have been verified by microbiological examination.” Emilson C G. Potential efficacy of chlorhexidine against mutans streptococci and human dental caries. J Dent Res 1994; 73: 682-691.

1.4 Side Effects

Considering the widespread use of chlorhexidine, very few complaints have been reported. Some local side effects do exist such as discoloration of teeth, tongue, restorations and dentures. Other untoward effects are soreness of the oral mucosa and temporary taste disturbances. The mentioned side effects are mostly related to the 0.2 percent mouthrinse.

Solutions containing SnF₂ show similar side effects as chlorhexidine concerning discoloration, but normally patients may complain about unpleasant metallic taste. These untoward effects are dependent upon the concentration.

Mouthrinses with increased concentration of hexetidine from 0.10 to 0.14% may show an increased desquamation. Ref: “Scheie A. Modes of action of currently known chemical antiplaque agents other than chlorhexidine. J Dent Res (Spec Iss) 1989; 68:1909-1616.

Anions

2.1 Substances, Vehicles and Concentrations

Fluoride containing substances are the dominating anionic agents. Fluoride has been utilized since 1940's because of its known caries-reducing effects, mainly by affecting demineralization/remineralization processes. Fluoride containing substances also exhibit some antimicrobial properties.

Example of another anionic agent is sodium lauryl sulphate. The substance is often used in commercial dentifrices as a surfactant.

2.2 Operation

Anionic agents compete with the negatively charged bacteria for the positively charged ligands. This leads to an inhibition of bacterial adhesion. The ligands are protein structures, for example in the pellicle. Also hydroxyapatite can act as a ligand. Fluoride can affect bacteria in different ways dependent on the concentration. Three general effects are seen.

There is an influence on the normal membrane function of the bacteria.

A positively charged glycolytic enzyme is affected by fluoride. The activity of the enzyme is lowered or inhibited which leads to a reduced emittance of acid end-products by the bacteria.

At a low pH the glucose uptake can be inhibited. This is caused by a reduced function of a membrane gradient for H⁺ which influences one of the two types of canals for glucose intake.

2.3 Efficacy

The use of fluoride containing substances for caries prevention is well documented regarding its total effects (for remineralization as well as adhesion- and metabolical inhibition). The duration of effect depends on the concentration of the agent but even on the selection of vehicle. Vehicles that give rise to inhibitory effects require a concentration of >100 μg/ml NaF. To be bacteriocidal a 30-fold increase in concentration is necessary. Vehicles containing less than 100 μg/ml NaF participate at least in the remineralization. (Values from Ekstrand J, Fejerskov O, Silverstone L M. Fluoride in dentistry. Copenhagen: Munksgaard, 1988). The surfactants show plaque inhibitory properties. Embery G, Rölla G. Clinical and biological aspects of dentifrices. Oxford: Oxford University Press, 1992.

2.4 Side effects

Normally there are no untoward effects for fluoride containing substances when used according to recommendations. For effects of high fluoride doses, see for example Ekstrand J, Fejerskov O, Silverstone L M. Fluoride in dentistry. Copenhagen: Munksgaard, 1988). Sodium lauryl sulphate is protein denaturating. This can result in epithelial desquamation. Fakhry Smith S, Din C, Nathoo S A, Gaffar A. Clearance of sodium lauryl sulphate from the oral cavity. J Clin Periodontol. 1997 May 24: 313-317.

Non-Ionic Agents

3.1 Substances, Vehicles and Concentrations

There are two types of phenol like substances. One is triclosan which is a non-charged agent and the other is Listerine® which is a combination of the phenol-related essential oils thymol and eucalyptol. (Thymol is also a constituent in the chlorhexidine varnish Cervitec). Triclosan is normally not the only antimicrobial agent in different vehicles. To augment the efficacy the surface coating copolymer, polyvinylmethyl ether and maleic acid, commercially known as Gantrez®, is added. The following products are examples of phenol like substances:

0.3 percentage of triclosan+copolymer—Colgate Total dentifrice, manufacturer: Colgate Palmolive

Thymol and eucalyptol—Listerine mouthrinse, manufacturer: Warner Lambert.

3.2 Operation

Agents belonging to this group are phenol derivatives, i.e. they exhibit both hydrophilic and hydrophobic properties and are non-ionic. The activity is of a broad spectrum character affecting a wide range of oral Gram positive and Gram negative bacteria. (Kjærheim V. Experiments with triclosan. Thesis, Univ. of Oslo, Norway 1995).

The exact mode of action is not fully understood, but the suggested way of action, which is dependent on the concentration or the agent.

The rapid uptake in the bacterial cell wall, leads to the inhibition of the membrane located enzymes PTS and PMF. The effect of this action is a reduced or inhibited uptake of glucose which leads to a less or none cellular acid production.

Higher concentrations cause membrane leakage and which leads to bacteriolysis.

3.3 Efficacy

Both triclosan and Listerine® have been used in antiseptic treatment related to the skin for many decades. From an oral perspective, the two phenol derivatives have been available and used since the end of the 80's. As mentioned earlier, triclosan is combined with Gantrez® just to enhance the inhibition of bacterial growth and proliferation of plaque bacteria. This makes triclosan slightly more effective compared to Listerine®. See: Embery G, Rölla G. Clinical and biological aspects of dentifrices. Oxford: Oxford University Press, 1992.

The efficacy is always governed by the concentration and substantivity. For the Gantrez® combination it seems that a minimum quotation of 0.3% triclosan/0.25% copolymer mouthrinse is needed to give a modest effect and a 0.3% triclosan/2% copolymer dentifrice is needed to give a moderate effect (values taken from “Experiments with triclosan” Kjærheim V. Thesis, Univ. of Oslo, Norway 1995).

Triclosan reduces skin reactions like epithelial desquamation caused of sodium lauryl sulphate. Barkvoll P, Roölla G. Triclosan protects the skin against dermatitis caused by sodium lauryl sulphate exposure. J Clin Periodontol 1994 Nov; 21: 717-719.

Compared to a chlorhexidine mouthrinse (Peridex) Listerine was less plaque reducing in a 14 day twice daily rinsing with 3 mouthrinses where the subjects were asked to refrain from all other oral hygiene procedures. Maruniak J, Clark W B, Walker C B, Magnusson I, Marks R G, Taylor M, Clouser B. The effect of 3 mouthrinses on plaque and gingivitis development. J Clin Periodontol 1992; 19: 19-23.

A triclosan/copolymer containing dentifrice showed in a six month clinical study a significant reduction in supragingival plaque compared to a triclosan/zinc containing dentifrice that showed no plaque reduction. Palomo F, Wantland L, Sanchez A, Volpe A R, McCool J, DeVizio W. The effect of three commercially available dentifrices containing triclosan on supragingival plaque formation and gingivitis: a six month clinical study. Int Dent J 1994; 44: 75-81.

Enzymes

4.1 Substances, Vehicles and Concentrations

Enzymes like amyloglucosidase and glucose oxidase are found as added ingredients in dentifrices and mouthrinses. Example of products are:

Amyloglucosidase and glucose oxidase—Zendium Pepparmint dentifrice, manufacturer: Meda Sverige AB

Amyloglucosidase and glucose oxidase—Zendium Munskölj mouthrinse, manufacturer: Meda Sverige AB

4.2 Operation

There are different types of enzymes that show antimicrobial activity in in vitro tests. One example is dextranase. This enzyme is believed to dissolve the linkage in the adhesin dextran (glucan), theoretically resulting in a reduced accumulation of dental plaque. Schilling K M, Bowen W H. Glucans synthesized in situ in experimental salivary pellicle function as specific binding sites for Streptococcus mutans. Infect Immun 1992; 90: 284-295.

A combination consisting of amyloglucosidase and glucose oxidase acts in a different manner. These two enzymes enhance the sialoperoxidase system, which is dependent on the metabolic end product hydrogen peroxide. Amyloglucosidase split amylase to glucose. Glucose oxidase produces hydrogen peroxide of glucose. The gist of the enzymes activity is to control the proliferation of bacteria by augmenting the presence of hypothiocyanite at neutral pH or hypothiocyanous acid at low pH. Hypothiocyanite is believed to enhance the lytic action of lysozyme. Based on Embery G, Rölla G. Clinical and biological aspects of dentifrices. Oxford: Oxford University Press, 1992.

4.3 Efficacy

Some studies have shown anti-plaque results of enzyme containing dentifrices, other studies failed to confirm this.

### SUMMARY OF THE INVENTION

While previous research has focused on modifying the activity of glycogen synthase or ADP-glucose pyrophosphorylase to modify starch content in plants, the present inventor has determined that, since the catabolism and metabolism of energy storage pathways are critical to viability of S. mutans, the inhibition of such pathways in S. mutans provides a novel class of antibiotics for the treatment of S. mutans infection. Further, numerous studies have indicated that glycogen plays an important role in the survival of the bacterial cell (Strange, R. E. (1968) Nature 220, 606; Strange et al (1961) J. Gen. Microbiol. 25, 61; Van Houte, J., and Jansen, H. M. (1970) J. of Bacteriol. 101, 1083). Whereas current antibiotics are characterized by inhibition of protein synthesis, DNA synthesis and cell wall synthesis, this novel class of antibiotics is characterized by inhibition of energy storage and utilization pathways.

The inventor has particularly noted that production of ADP-glucose is critical to viability of S. mutans, and since ADP-glucose pyrophosphorylase is not present in mammals, the enzyme provides an excellent target for inhibition of S. mutans growth, thereby providing a means for inhibiting the growth of S. mutans and treating S. mutans infection.

Similarly, since glycogen synthase in S. mutans is different than the glycogen synthase in mammals, that enzyme also provides an excellent target for inhibition of S. mutans growth and treating S. mutans infection.

In one aspect, the present invention is directed to a method for treating a microorganism infection by administering an effective amount of a compound capable of inhibiting the production and/or utilization of ADP-glucose.

It is another aspect of the invention to provide a method for treating a microorganism infection by administering an effective amount of a compound capable of inhibiting the activity of ADP-glucose pyrophosphorylase and/or glycogen synthase.

It is a further aspect of the invention to provide a method of identifying a compound capable of inhibiting the growth of pathogenic microorganisms which comprises identifying a compound which inhibits an enzyme important in the catabolism and metabolism of energy storage pathways, particularly a compound that inhibits the activity of ADP-glucose pyrophosphorylase and/or glycogen synthase.

The present inventor has identified the bacteria S. mutans as having the ADP-glucose pyrophosphorylase pathway for energy storage and utilization.

Moreover, the present inventor has determined that antimicrobial agents that inhibit the activity of ADP-glucose pyrophosphorylase and/or glycogen synthase will inhibit the growth of S. mutans or render S. mutans non-infective/pathogenic.

In addition, the present inventor has determined that inhibitors of ADP-glucose pyrophosphorylase and/or glycogen synthase when added to toothpaste or mouthwash will inhibit the growth of S. mutans or render S. mutans non-infective/pathogenic.

In one aspect, the present invention is directed to a method for treating S. mutans infection by adding to toothpaste or mouthwash an effective amount of a compound capable of inhibiting the production and/or utilization of ADP-glucose.

It is another aspect of the invention to provide a method for treating S. mutans infection by adding to toothpaste or mouthwash an effective amount of a compound capable of inhibiting the activity of ADP-glucose pyrophosphorylase and/or glycogen synthase.

It is a further aspect of the invention to provide a method of identifying a compound capable of inhibiting the growth of S. mutans which comprises identifying a compound which inhibits an enzyme important in the catabolism and metabolism of energy storage pathways, particularly a compound that inhibits the activity of ADP-glucose pyrophosphorylase and/or glycogen synthase.

### BRIEF DESCRIPTION OF THE FIGURES

FIGS. 1-5 are graphs plotting the results of experiments testing the inhibiting effect of ADP-glucose borano analogs on an ADP-glucose Ppase enzyme in in vitro enzyme tests.

### DETAILED DESCRIPTION OF THE INVENTION

The present inventor has discovered that while certain important pathogenic microorganisms require the activity of ADP-glucose pyrophosphorylase (EC 2.7.7.27) to produce ADP-glucose, that enzyme is not present in mammals, particularly not in humans. In addition, the present inventor has recognized that the glycogen synthase (EC 2.4.1.21) in many pathogenic bacteria as the ADPGlucose dependent glucan chain lengthening enzyme is not present in mammals, particularly not in humans. As a result, the present inventor has first determined that inhibition of ADP-glucose pyrophosphorylase (EC 2.7.7.27) and/or glycogen synthase (EC 2.4.1.21) provide excellent targets for inhibiting the growth of pathogenic microorganisms, while not inhibiting any important biosynthetic pathway in humans.

As noted above, in plants and most bacteria, ADP-glucose pyrophosphorylase catalyzes the reaction of α-glucose-1-phosphate with ATP to produce ADP-glucose. In mammals and in eukaryotic microorganisms, the “corresponding” reaction is catalyzed by UDP-glucose pyrophosphorylase by transferring a glucosyl residue from UDP-glucose, as shown below:

(1) UTP+glucose-1-phosphate→UDP-glucose (UDPGlc)+PPi

(2) UDPGlc+α-1,4-glucan→UDP+α-1,4-glucosyl-α-1,4-glucan.

Step (1) in this pathway in mammals, including humans, is catalyzed by UDP-glucose pyrophosphorylase. Recognizing the difference between the critical use of UDPGlc PPase in mammals as compared to the use of ADPGlc PPase in certain pathogenic bacteria, the present inventor first recognized that such bacteria could be selectively killed by inhibiting the activity of ADPGlc PPase, without adversely affecting any mammal so infected with the bacteria. This selectivity provides a basis and target for a novel and important new class of antibiotics, antibiotics which are inhibitors of energy storage and utilization enzymes, namely ADPGlc PPase inhibitors.

Step (2) in the above is catalyzed in humans by glycogen synthase (EC 2.4.1.1 1), whereas in many pathogenic bacteria the corresponding enzyme is EC 2.4.1.21 because the sugar substrate is ADP-based, not UDP-based. Again, this provides a target for a novel and important new class of antibiotics which inhibit glycogen synthase (EC 2.4.1.21).

As is well known, antibiotics are currently used to treat a wide range of bacterial infections, ranging from minor to life threatening infections. Broad spectrum antibiotics treat a variety of gram-positive and gram-negative organisms, while mild spectrum antibiotics only cover limited types of bacterial organisms and are useful for curing infections with known bacterial strains.

But it has recently been noted that pathogenic bacteria and fungi increasingly exhibit resistance to existing classes of antibiotics, such as penicillin, vancomycin and erythromycin.

According to the Center for Disease Control, pathogenic resistance has significantly increased mortality rates, making infectious disease the third largest cause of death in the United States. The rates of antibiotic resistant bacteria have particularly increased recently with respect to S. aureus, Enterococcus strains, S. pneumoniae and M. tuberculosis.

The mechanism of action for most antibiotics is the inhibition of bacterial cell wall completion, or DNA or protein synthesis. Sulfonamides and trimethoprin act by inhibiting an essential metabolic step, namely folate synthesis. But there is a great need for new antibiotics with different targets, especially in light of the ever increasing problem of resistant strains.

The present inventor has found that compounds which act as inhibitors of ADP-glucose pyrophosphorylase and/or glycogen synthase offer another class of antibiotics which inhibit an essential bacterial metabolic step, namely a pathway essential for bacterial energy storage and utilization. A first aspect of the invention relates to a method for identifying compounds capable of inhibiting the growth of S. mutans which comprises:

a. identifying an enzyme that is important to energy storage or utilization in S. mutans, which enzyme is not present in mammals, particularly not in humans; and

b. identifying a compound that inhibits that enzyme in S. mutans.

c. adding said compound to toothpaste or mouthwash to inhibit the growth of S. mutans or render S. mutans non-infective/pathogenic.

According to the present invention, an enzyme in an energy storage or utilization pathway which is important to continued growth and viability of S. mutans but which is absent in humans provides a unique, specific target for compounds which can inhibit infections of S. mutans without causing undesirable side effects or toxicity to a mammalian patient. Various biosynthetic pathways have been identified in the literature for various microorganisms and for mammals, and those pathways, which include an important enzyme present in pathogenic microorganisms but absent in mammals, provide a unique target for screening for compounds useful for inhibiting pathogenic microorganism infections.

According to the present invention, the present inventor has specifically identified ADP-glucose pyrophosphorylase (EC 2.7.7.27) and glycogen synthase (EC 2.4.1.21) as enzymes present in a biosynthetic pathway important for energy storage and utilization in S. mutans, but absent in mammals, specifically absent in humans. Since the biosynthetic pathway is important for energy storage or utilization in S. mutans, inhibition of this pathway significantly decreases the viability of S. mutans, leading ultimately to death of S. mutans, either by action of the inhibitor alone, or in combination with the patient's own immunological systems for resisting infections, or in combination with other antibiotics.

In some instances, inhibition of the biosynthetic pathways and/or inhibition of ADP-glucose pyrophosphorylase (EC 2.7.7.27) or glycogen synthase (EC 2.4.1.21) may not per se kill the bacteria, but will render the microorganisms non-infective or non-pathogenic. It has been known for quite some time that complex carbohydrates can act as virulence factors in bacteria responsible for invasive infections (Glazer, A. N. and Nikaido, H. Microbial Biotechnology. Fundamentals of Applied Microbiology (1995) W. H. Freeman and Co., pgs. 266-272). Specific to glycogen biosynthesis, Uttaro, A. D., and Ugalde, R. A. Gene 150: 117-122 (1994) and J. E. Ugalde et al., J. Bact. 180: 6557-64 (1998) have shown that mutations that prevent gene expression of the gig operon render Agrobacterium tumefaciens non-infective. Illiffe-Lee and McClarty, Mol. Microbiology 38(1): 20-30 (2000) have shown that growth of Chlamydia trachomatis, under conditions that limit glycogen, severely reduces the number of infectious bodies.

Description of Method for Inhibition Screening

As noted above, one aspect of the present invention is a method for the identification of a compound capable of inhibiting the growth of S. mutans by interfering with the activity of glycogen synthase and/or ADP-glucose pyrophosphorylase. Compounds can be identified by growing bacteria on defined media in the presence or absence of a test compound, and assessing the effect on glycogen synthesis by iodine staining of colonies (Govons, S. et al., (1969) J. Bacteriol. 97: 970-972). More quantitatively, the amount of glucan accumulated in the absence or presence of test compounds can be assessed by collection of the glycogen from the culture and quantitatively converting it to glucose with glucoamylase and α-amylase (Preiss, J. et al., (1975) J. Biol. Chem. 250: 7631-7638)

Compounds capable of inhibiting glycogen synthase and/or ADP-glucose pyrophosphorylase can also be identified by means of in vitro experiments by exposing a substrate comprising glycogen synthase and/or ADP-glucose pyrophosphorylase to a plurality of test compounds and identifying those compounds which inhibit the tested enzyme according to known catalytic measurement techniques.

One particular in vitro method for assessing the activity of an inhibitor to purified ADPGlc PPase is the following:

Assaying for ADPGlc synthesis activity involves measuring the amount of 14-C labeled Glc-1-P converted to ADPGlucose (Preiss, J., Shen, L., Greenberg, E., and Gentner, N. (1966) Biochemistry 5, 1833-1845). Briefly, unreacted Glc-1-P is separated from product by the following steps: 1)digestion with alkaline phosphatase (thus removing the negatively charged phosphate); 2) spotting an aliquot of the reaction mixture on to positively charged DE-81 filters (Whatman); and 3) washing the filters with water (thus removing the now neutral C-14 glucose).

To assess the effect of a putative inhibitor, enzyme assays are performed at a subsaturating concentration of substrate (depending on the enzyme, ATP=0.2-1 mM, Glc-1-P=0.5 mM) under standard conditions in the absence and presence of the major activator for each enzyme (1-5 mM depending on enzyme). In this way, the effect of the inhibitors can be evaluated under the range of the expected in vivo conditions. Initial screening of a putative inhibitor typically includes testing at two concentrations (˜25 μM and 1 mM)±major activator with appropriate controls and blanks for a total of 9 assays/inhibitor/enzyme (2 control assays—appropriate enzyme concentrations in the absence of inhibitor; 4 experimental assays; 1 blank in the absence of inhibitors; 2 blanks in the presence of inhibitor (no activator required).

A quantitative assay to measure the activity of an inhibitor to bacterial (ADPG dependent) Glycogen Synthase (EC 2.4.1.21) involves the measurement of incorporated 14-C-labeled ADPGlc into a glucan molecule (a specific glycogen, or amylopectin) that serves as a primer wherein the labeled glucan can easily be separated from unincorporated ADPGlc by a precipitation step (methanol insoluble polysaccaride). Useful specific procedures are as described in Furakawa, K., Tagaya, M., Inouye, M., Preiss, J., and Fukui, T. (1990)

“Identification of Lys-15 at the Active Site in E. coli Glycogen Synthase,” J. Biol. Chem. 265, 2086-2090; and Thomas, J. A., Schlender, K. K., and Larner, J. (1968) Anal. Biochem. 25, 486-499.

Useful inhibitors can also be identified, and potential inhibitors assessed, by in vitro treatment of bacteria in, for example, culture tubes or petri dish samples. Such assessments can be performed, for example, by spreading a measured aliquot of a diluted bacterial culture into nutrient agar plates, both treated and control, and counting the number of visible cells. Detailed procedures are well known to those in the art, as shown for example in Miller, Experiments in Molecular Genetics, Cold Spring Harbor Laboratory, 1972.

Compounds which inhibit ADPGlc PPase or glycogen synthase can also be assessed in an in vivo animal model test. Such tests can be conducted in an animal which is susceptible to infection by the pathogenic microorganism of interest. For example, the effect of the test compound on the virulence of H. influenzae is assessed by comparing the survival rates of animals which have been administered the test compound with the survival rate of animals which have not been administered the test compound, wherein a higher survival rate of animals administered the test compound is an indication that the test compound has an effect on the virulence of H. influenzae.

To determine the effect of a test compound on colonization of the mucosal surface or on invasiveness and/or virulence of H. influenzae, the test compound is administered to the animals either prior to, at the time of, or after inoculation of the animals with H. influenzae. The test compound may be administered directly into the nasopharynx, or may be administered by any other route including any one of the traditional modes (e.g., orally, parenterally, transdermally or transmucosally), in a sustained release formulation using a biodegradable biopolymer, or by on-site delivery using micelles, gels and liposomes, or rectally (e.g., by suppository or enema). Precise formulations and dosages will depend on the nature of the test compound and may be determined using standard techniques, by a pharmacologist of ordinary skill in the art.

There are essentially two types of in vivo models in which a compound may be tested for antibiotic activity directed against S. pneumoniae. In the first model, colonization of the mucosal surface of the nasopharynx by S. pneumoniae in the presence or absence of the test compound is assessed. Measurement of colonization of the mucosal surface of the nasopharynx by S. pneumoniae may be conducted in an animal model essentially as described in Weiser et al. (1994, supra).

To assess colonization, briefly, an amount of S. pneumoniae, generally about 10 mu.l of phosphate buffered saline (PBS)—washed mid log phase organisms adjusted to the desired density, is inoculated into the left anterior naris of the animal. Colony counts are performed to ensure that the inocula are of the desired density and phenotype. The nasopharynx is cultured for the presence of viable S. pneumoniae by the slow instillation of 20 to 40 μl of sterile PBS into the left naris and withdrawal of the initial 10 μl from the right naris. This procedure ensures that the fluid has passed through the nasopharynx. The quantity of organisms recovered is then assessed in a well known culture assay.

The effect of the test compound on colonization of the nasopharynx by S. pneumoniae is evaluated by comparing the degree of colonization of the nasopharynx in animals which have been administered the test compound with the degree of colonization in animals which have not been administered the test compound, wherein a lower degree of colonization in animals administered the test compound is an indication that the test compound inhibits colonization of the nasopharynx by S. pneumoniae.

The invasive capability of S. pneumoniae may also be measured in the same animal model. Essentially, bacteria which have entered the blood stream following inoculation of the nasopharynx are detected by culturing the same in a sample of blood obtained from the animal. Again, the effect of the test compound on the invasive capacity of S. pneumoniae is assessed by comparing the number of organisms found in the blood stream in animals which have been administered the test compound with the number of organisms in the blood stream in animals which have not been administered the test compound, wherein a lower number of organisms in the blood stream of animals administered the test compound is an indication that the test compound has an effect on the invasive capacity of S. pneumoniae.

In a second in vivo model, the virulence of S. pneumoniae is assessed in animals as described in Berry et al. (1995, Infect. Immun. 63:1969-1974). Essentially, cultures of S. pneumoniae are diluted to a density of 2.times.10.sup.6 colony forming units per ml, and volumes of 0.1 ml are injected intraperitoneally into groups of animals. The survival time of the animals is recorded and the differences in median survival time between groups may be analyzed by the Mann-Whitney U test (two-tailed). Differences in the overall survival rate between groups may be analyzed by the x.sup.2 test (two tailed).

The effect of the test compound on the virulence of S. pneumoniae is assessed by comparing the survival rates of animals which have been administered the test compound with the survival rate of animals which have not been administered the test compound, wherein a higher survival rate of animals administered the test compound is an indication that the test compound has an effect on the virulence of S. pneumoniae.

To determine the effect of a test compound on colonization of the mucosal surface or on invasiveness and/or virulence of S. pneumoniae, the test compound is administered to the animals either prior to, at the time of, or after inoculation of the animals with S. pneumoniae. The test compound may be administered directly into the nasopharynx, or may be administered by any other route including any one of the traditional modes (e.g., orally, parenterally, transdermally or transmucosally), in a sustained release formulation using a biodegradable biopolymer, or by on-site delivery using micelles, gels and liposomes, or rectally (e.g., by suppository or enema). Precise formulations and dosages will depend on the nature of the test compound and may be determined using standard techniques, by a pharmacologist of ordinary skill in the art.

The present invention further provides a method for treating S. mutans infection in a patient by administering to the patient an effective amount of an inhibitor against glycogen synthase and/or ADP-glucose pyrophosphorylase, wherein an effective amount of the inhibitor will inhibit the activity of the enzyme so as to decrease viability of and/or kill the microorganism. The inhibitor utilized in the treatment may be one identified by one of the methods described above, or inhibitors may be identified by any other method. One such inhibitor is the nucleoside α-P-boranodiphosphoglucose (particularly its triethylammonium salt), which is a borane analog of glucose-conjugated nucleoside diphosphate, described by Lin and Shaw, Tetrahedron Letters 41 (2000) 6701-6704. The particular compound, adenosine α-P-boranodiphosphoglucose, is a boran analog of ADP-glucose generating two sterioisomers and has the following structures:

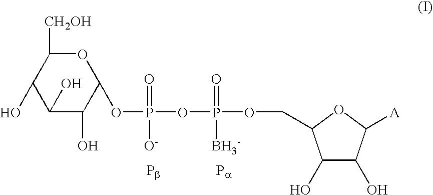

wherein A is adenosine.

Adenosine boranophosphate exists as two stereoisomers, the preparation of which is described below:

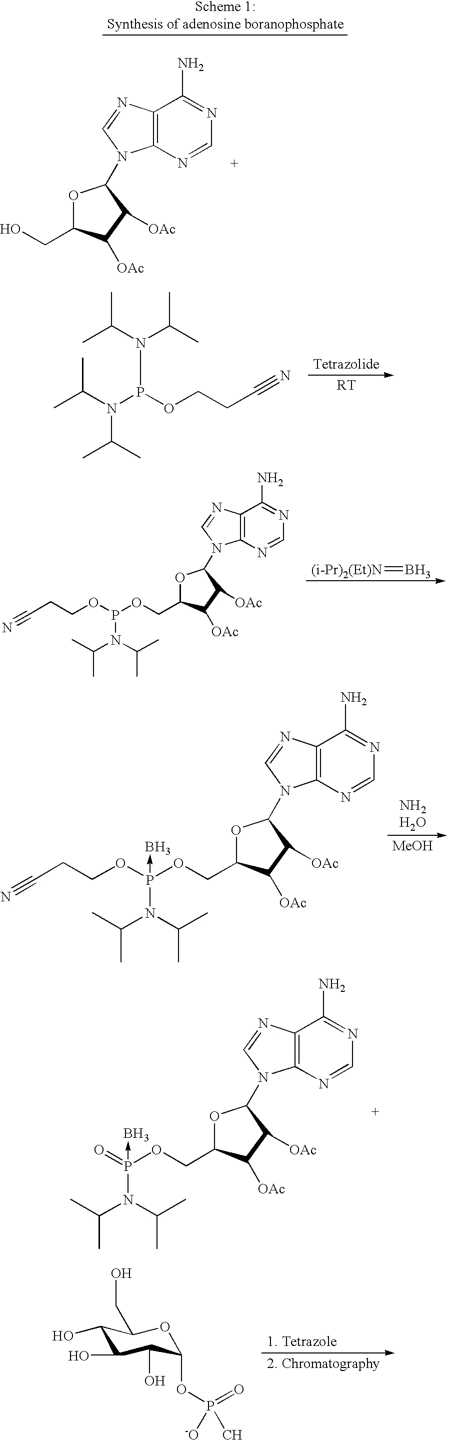

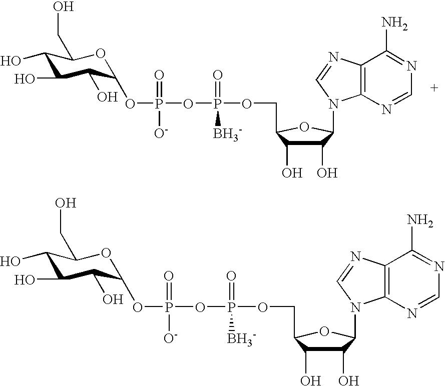

As an analog of ADP-glucose, the compound of formula (I) is a useful inhibitor of ADPGlc PPase according to the present invention.

Other known regulators and inhibitors of ADPGlc PPase are the following:

TABLE 1
Regulatory properties of ADP-glucose pyrophosphorylase in different
organisms

| | ADP-glucose pyrophosphorylase | |
|---|---|---|
| | Allosteric regulators | |
| Organism | Activator | Inhibitor |
| Prokaryotes | | |
| Enterobacteria | | |
| Escherichia coil | Fructose-1,6-bisP | AMP |
| Salmonella typhimurium | | |
| Enterobacter aerogenes | | |
| Aeromonas formicans | Fructose-1,6-bisP | AMP |
| Micrococcus luteus | Fructose-6-P | ADP |
| Mycobacterium smegmatis | | |
| Serratia mercescens | None | AMP |
| Enterobacter hafniae | | |
| Clostridium pasteurianum | | |
| Agrobacterium tumefaciens | Pyruvate | AMP |
| Arthrobacter viscosus | Fructose-6-P | ADP |
| Chromatium vinosum | | |
| Rhodobacter capsulata | | |
| Rhodomicrobium vannielli | | |
| Rhodobacter gelatinosa | Pyruvate | AMP |
| Rhodobacter globiformis | Fructose-6-P | Pi |
| | | (inorganic phosphate) |
| Rhodobacter sphaeroides | Fructose-1,6-bisP | |
| Rhodospirillum rubrum | Pyruvate | |
| Rhodospirillum tenue | | |
| Rhodocyclus purpureus | | |
| Cyanobacteria | 3-P-glycerate | Pi |
| Synechococcus 6301 | | |
| Synechocysits 6803 | | |
| Anabaena 7120 | | |
| Eukaryotes | 3-P-glycerate | Pi |
| Green algae | | |
| Chlorella fusca | | |
| Chlorella vulgaris | | |
| Chlamydomonas reinhardtii | | |
| Higher Plants | | |
| Photosynthetic tissues | 3-P-glycerate | Pi |
| (leaves of spinach, | | |
| Arabidopsis, wheat, maize, | | |
| rice) | | |
| Non-photosynthetic tissues | 3-P-glycerate | Pi |
| (Potato tubers, maize | | |
| endosperm) | | |

### EXAMPLE 1

Effect of ADPG Borano Analogs on Rhodobacter Spaeroides (RB.S.) ADPG Ppase

The APDGlc Ppase from Rhodobacter sphaeroides has been used as a model enzyme for initial testing of inhibitors as it has been cloned, sequenced, and expressed in the inventor's laboratory (Meyer et al (1999) Arch. Biochem. Biophys. 372, 179-188; Igarashi, R. Y. and Meyer, C. R. (2000) Arch. Biochem. Biophys. 376, 47-58) and the purified recombinant enzyme is readily available. Further, this particular enzyme has the most complex regulation (having partial overlap with nearly every type of ADPG Ppase) and has sufficient homology (ranging from ˜40-70% similarity) to the enzyme primary sequence from the mentioned pathogenic bacteria.

1. OVERVIEW: 2 analogs (ANALOG; stereoisomers I, II) of adenosine-α-P-boranodiphosphoglucose were tested for inhibiting activity against the ADPG Ppase enzyme from Rhodobacter sphaeroides (Rb.s.) ADPG Ppase.

2. MATERIALS

PREMIX: A: 100 mM HEPES (pH 7), 0.5 mg/mL BSA, 0.5 mM Glc-1-P, 0.25 mM ATP subsaturating (near S0.5 value), 5 mM MgCl2, (0.2 U Pyrophosphatase))

PREMIX B: A+1 mM F6P (170 μL premix will allow 20 μL for analog vol., 10 μL of E)

II. ANALOG CONC.=5, 50, 500 μM (STOCK CONC.=5 mM) in 50 mM HEPES, pH 7, note that 20 μL of 5 mM in 200 μL assay will yield 500 μM final concentration to obtain 5 mM aliquots: ANALOG I: add 625 μL of HEPES buffer, mix thoroughly ANALOG II: add 500 μL of HEPES buffer, mix thoroughly

1:10 DILUTION ALIQUOTS OF 5 mM STOCKS are prepared in order to generate stock solutions that will give 50 and 5 μM with the addition of 20 μL. 100 μL aliquots are stored at −20° C.

RBS ADPG PPASE WT ENZYME CONC.=17 mg/mL (use 1:8000 dilution in Dilution Buffer)

THE FOLLOWING ASSAY SET OF 11 ARE UTILIZED FOR EACH ANALOG

1-BLANK (Premix A)

2-BLANK+50 μM ANALOG (A)

3-BLANK+500 μM ANALOG (A)

4-E CON (A)

5-E CON+F6P (B)

6-E+5 μM (A)

7-E+5 μM (B)

8-E+50 μM(A)

9-E+50 μM(B)

10-E+500 μM (A)

11-E+500 μM (B)

Purified Rb.s ADPG Ppase was assayed under standard conditions (0.25 mM ATP [˜S0.5 value], 0.5 mM Glc-1-P, 5 mM Mg, 100 mM HEPES, pH7) in the presence and absence of the activator F6P (1 mM) at 3 concentrations of the analogs: 5, 50, and 500 μM. Analog stock solutions were made in 50 mM HEPES, pH 7). These concentrations should be regarded as estimates as there could have been some breakdown of the compounds during 2 years of storage at −20° C. The analogs had no effect on the background cpm of the assays.

Results

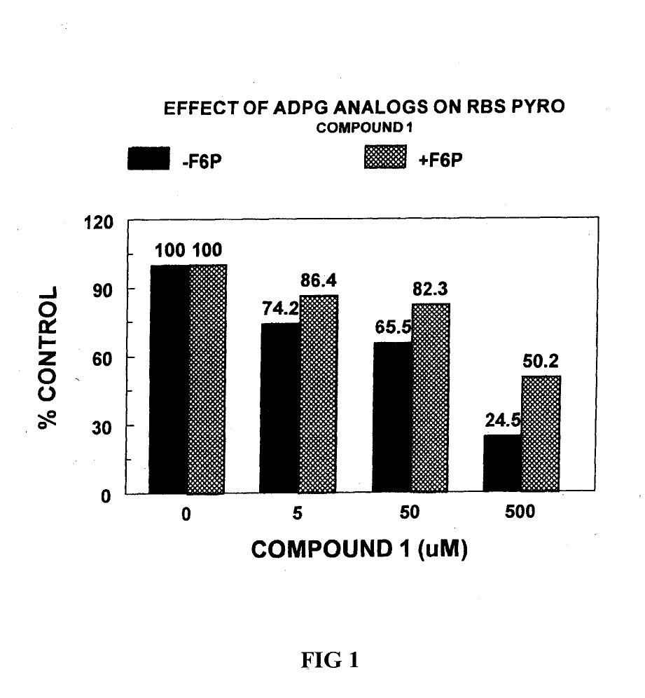

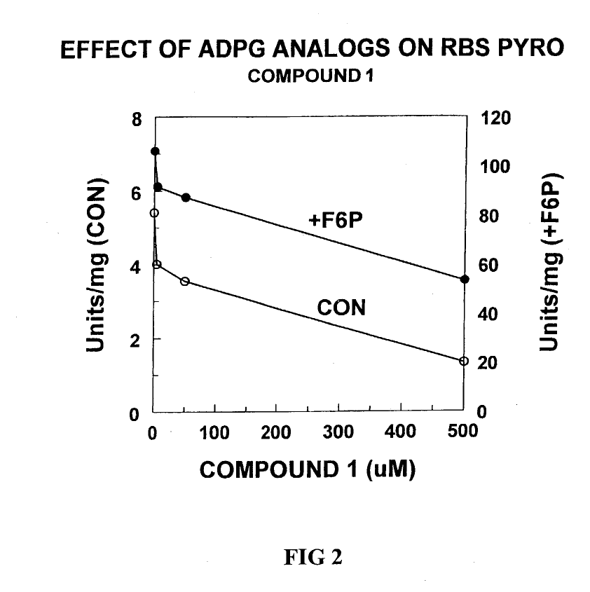

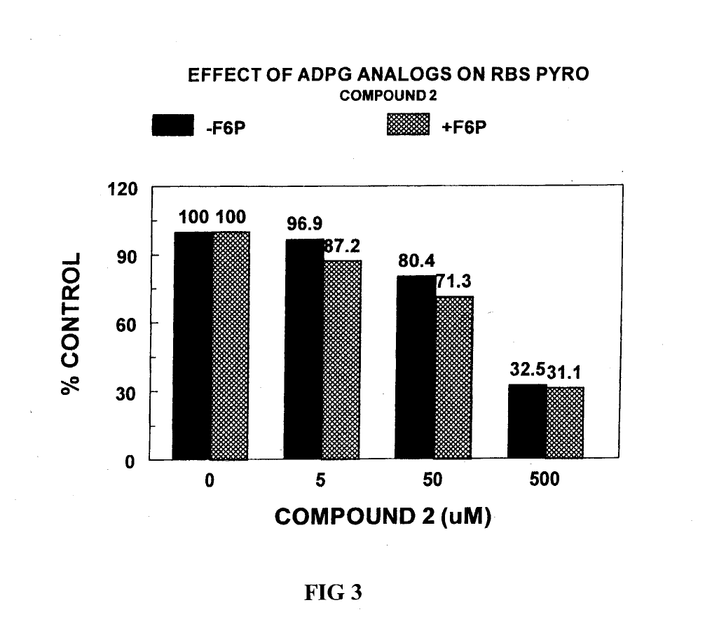

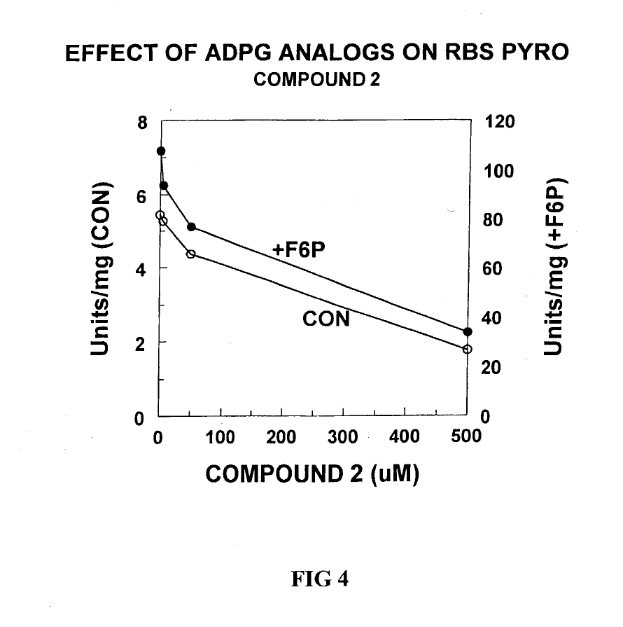

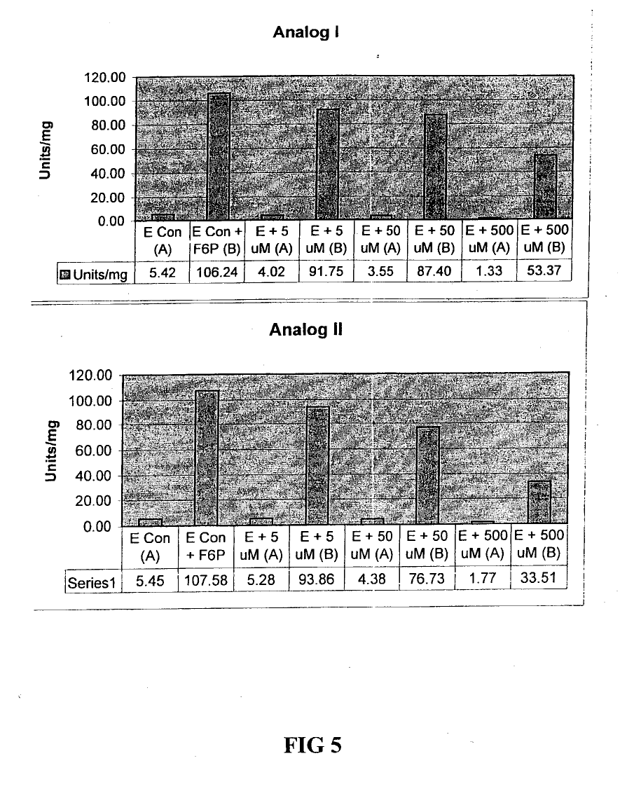

The detailed experimental data results are set forth in Table 3, and those results are graphed in FIGS. 1-5.

Analog I successfully inhibited the Rb.s. ADPG Ppase. While the response may be biphasic, the results showed about 75% inhibition by 500 μM in the absence of the activator F6P. The inhibition was less in the presence of F6P (˜50% at 500 μM). Analog II also successfully inhibited the Rb.s. enzyme with about 70% inhibition at 500 μM in both the presence and absence of F6P.

TABLE 2
Inhibition Study with ADPGlucose Borano compounds Analog I and II

| | Analog I | Analog II |
|---|---|---|
| Blank | 60.33 | 56.33 |
| Blank + 50 uM | 76.33 | 51.00 |
| Blank + 500 uM | 53.67 | 53.33 |
| | 825 Cpm/nmol | |

| | | Volume | | | | | | | dil. Corr | |
|---|---|---|---|---|---|---|---|---|---|---|
| Description | Dil | (μL) | CPM | cor. CPM | nmol | *2.2 | nmol/min | nmol/min/uL | nmol/min/uL | Units/mg |
| E Con (A) | 8000 | 10 | 492.00 | 431.67 | 0.52 | 1.151 | 0.115 | 0.0115 | 92.090 | 5.42 |
| E Con + F6P (B) | 8000 | 10 | 8526.00 | 8465.67 | 10.26 | 22.575 | 2.258 | 0.2258 | 1806.010 | 106.24 |
| E + 5 uM (A) | 8000 | 10 | 380.67 | 320.34 | 0.39 | 0.854 | 0.085 | 0.0085 | 68.339 | 4.02 |
| E + 5 uM (B) | 8000 | 10 | 7371.33 | 7311.00 | 8.86 | 19.496 | 1.950 | 0.1950 | 1559.680 | 91.75 |
| E + 50 uM (A) | 8000 | 10 | 359.33 | 283.00 | 0.34 | 0.755 | 0.075 | 0.0075 | 60.373 | 3.55 |
| E + 50 uM (B) | 8000 | 10 | 7041.00 | 6964.67 | 8.44 | 18.572 | 1.857 | 0.1857 | 1485.796 | 87.40 |
| E + 500 uM (A) | 8000 | 10 | 160.00 | 106.33 | 0.13 | 0.284 | 0.028 | 0.0028 | 22.684 | 1.33 |
| E + 500 uM (B) | 8000 | 10 | 4306.67 | 4253.00 | 5.16 | 11.341 | 1.134 | 0.1134 | 907.307 | 53.37 |
| E Con (A) | 8000 | 10 | 491.00 | 434.67 | 0.53 | 1.159 | 0.116 | 0.0116 | 92.730 | 5.45 |
| E Con + F6P (B) | 8000 | 10 | 8629.00 | 8572.67 | 10.39 | 22.860 | 2.286 | 0.2286 | 1828.836 | 107.58 |
| E + 5 uM (A) | 8000 | 10 | 477.00 | 420.67 | 0.51 | 1.122 | 0.112 | 0.0112 | 89.743 | 5.28 |
| B + uM (B) | 8000 | 10 | 7536.00 | 7479.67 | 9.07 | 19.946 | 1.995 | 0.1995 | 1595.663 | 93.86 |
| E + 50 uM (A) | 8000 | 10 | 399.67 | 348.67 | 0.42 | 0.930 | 0.093 | 0.0093 | 74.383 | 4.38 |
| E + 50 uM (B) | 8000 | 10 | 6165.33 | 6114.33 | 7.41 | 16.305 | 1.630 | 0.1630 | 1304.390 | 76.73 |
| E + 500 uM (A) | 8000 | 10 | 194.33 | 141.00 | 0.17 | 0.376 | 0.038 | 0.0038 | 30.080 | 1.77 |
| E + 500 uM (B) | 8000 | 10 | 2724.00 | 2670.67 | 3.24 | 7.122 | 0.712 | 0.0712 | 569.743 | 33.51 |

Inhibitors useful for the treatment of S. mutans can be administered by a variety of means and dosage forms well known to those skilled in the art. When used as an antimicrobial agent in the treatment of microorganism infections, the present compounds are administered, for example, orally in the form of a tablet, capsule, powder, syrup, liquid, emulsion, paste. For the treatment, the inhibitor(s) can be administered in the form of a mouthwash, in a toothpaste, in a gum, in a lozenge or in any other form capable of administering/applying the inhibitor(s) to the dental areas affected by the S. mutans infection.

The suitable administration forms as mentioned above may be prepared by mixing an active ingredient with a conventional pharmaceutically acceptable carrier, excipient, binder, stabilizer, etc. When administered in the form of an injection, a pharmaceutically acceptable buffering agent, solubilizer, isotonic agent, etc. may be added thereto. The active compound may be administered per se, or in the form of a pharmaceutically acceptable salt thereof, or in the form of a pro-drug, such as an ester.

The dosage of the compound varies according to the conditions, ages, weights of the patient, the administration form, the frequency of the administration, etc., but it is usually in the range of 100 to 3000 mg per day for an adult, which is administered once or divided into several dosage units.

All of the publications referred to herein, are hereby specifically incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed:

1. A method of treating an S. mutans infection in a patient which comprises administering to said patient an effective amount of a compound capable of inhibiting an enzyme that is important to energy storage or utilization in said microorganism.

2. A method of treating an S. mutans infection in a patient which comprises administering to said patient an effective amount of a compound capable of inhibiting the production of ADP-glucose.

3. A method of treating an S. mutans infection in a patient which comprises administering to said patient an effective amount of a compound capable of inhibiting the conversion of α-glucose-1-phosphate+ATP into ADP-glucose+Ppi.

4. A method of treating an S. mutans infection in a patient which comprises administering to said patient an effective amount of a compound capable of inhibiting the chain elongation of ADP glucose.

5. A method of treating an S. mutans infection in a patient which comprises administering to said patient an effective amount of a compound to inhibit the activity of glycogen synthase (EC 2.4.1.21).

6. A method of treating an S. mutans infection in a patient, which comprises administering to said patient an effective amount of a compound capable of inhibiting the activity of ADP glucose pyrophosphorylase (EC 2.7.7.27).

7. The method according to claim 1, wherein said patient is a human.

8. The method according to claim 1, wherein administering to said patient consists of adding compound to toothpaste or mouthwash.

9. The method according to claim 1, wherein said compound is adenosine boranodiphosphoglucose, or a pharmaceutically acceptable salt thereof.

10. A pharmaceutical composition for the treatment of an S. mutans infection which comprises a pharmaceutically acceptable carrier and an effective anti-microbial amount of adenosine boranodiphosphoglucose, or a pharmaceutically acceptable salt thereof.

11. A pharmaceutical composition for the treatment of an S. mutans infection which comprises a pharmaceutically acceptable carrier and an effective anti-microbial amount of a compound which is an inhibitor of ADP-glucose pyrophosphorylase (EC 2.7.7.27), or a pharmaceutically acceptable salt thereof.

12. A pharmaceutical composition for the treatment of an S. mutans infection which comprises a pharmaceutically acceptable carrier and an effective anti-microbial amount of a compound which is an inhibitor of glycogen synthase (EC 2.4.1.21), or a pharmaceutically acceptable salt thereof.

13. The pharmaceutical composition according to claim 11, wherein said compound is adenosine boranodiphosphoglucose.

14. A method of identifying a compound capable of inhibiting the growth of S. mutans in a mammalian patient, which comprises:
a) identifying an enzyme that is important to energy storage or utilization in said pathogenic microorganism, which enzyme is not present in said mammalian patient; and
b) identifying a compound that inhibits said enzyme in said pathogenic microorganism.
c) adding said compound to toothpaste or mouthwash.

15. The method according to claim 14, wherein said mammalian patient is a human patient.

16. A method of identifying a compound capable of rendering S. mutans non-infective/pathogenic in a mammalian patient, which comprises:
a) identifying an enzyme that is important to energy storage or utilization in said pathogenic microorganism, which enzyme is not present in said mammalian patient; and
b) identifying a compound that inhibits said enzyme in said pathogenic microorganism.
c) adding said compound to toothpaste or mouthwash.

17. The method according to claim 16, wherein said mammalian patient is a human patient.

18. A method of identifying a compound capable of inhibiting the growth of S. mutans which comprises identifying a compound that inhibits the conversion of α-glucose-1-phosphate +ATP into ADP-glucose+Ppi.

19. A method of identifying a compound capable of inhibiting the growth of S. mutans which comprises identifying a compound that inhibits the chain elongation of ADP glucose.

20. A method of identifying a compound capable of inhibiting the growth of S. mutans by interfering with energy storage or utilization in said microorganism which comprises identifying a compound that inhibits the activity of ADP glucose pyrophosphorylase (EC 2.7.7.27).

21. A method of identifying a compound capable of inhibiting the growth of S. mutans by interfering with energy storage or utilization in said microorganism which comprises identifying a compound that inhibits the activity of glycogen synthase (EC 2.4.1.21).

22. A method of identifying a compound capable of inhibiting the growth of S. mutans by interfering with the activity of ADP-glucose pyrophosphorylase (EC 2.7.7.27) which method comprises incubating a sample of bacteria in a media in the presence or absence of a test compound, and assessing the effect on conversion of α-glucose-1-phosphate, wherein a lower level of conversion of α-glucose-1-phosphate in the presence of said test compound, compared with the level of conversion of α-glucose-1-phosphate in the absence of said test compound, indicates that said test compound interferes with the activity of ADP glucose pyrophosphorylase (EC 2.7.7.27).

23. A method of identifying a compound capable of inhibiting the growth of S. mutans by interfering with the activity of glycogen synthase (EC 2.4.1.21) which method comprises incubating a sample of bacteria in a solution containing a known amount of ADP glucose in the presence or absence of a test compound, and assessing the effect on chain elongation of ADP glucose in the presence of said test compound, compared with the level of chain elongation in the absence of said test compound, indicates that said test compound interferes with the activity of glycogen synthase (EC 2.4.1.21).

24. A method of identifying a compound capable of inhibiting the growth of S. mutans by interfering with the activity of ADP glucose pyrophosphorylase (EC 2.7.7.27) which method comprises exposing a substrate comprising ADP glucose pyrophosphorylase (EC 2.7.7.27) to a plurality of test compounds and identifying a test compound which binds to said ADP glucose pyrophosphorylase (EC 2.7.7.27).

25. A method of identifying a compound capable of inhibiting the growth of S. mutans by interfering with the activity of glycogen synthase (EC 2.4.1.21) which method comprises exposing a substrate comprising glycogen synthase (EC 2.4.1.21) to a plurality of test compounds and identifying a test compound which binds to said glycogen synthase (EC 2.4.1.21).

26. The method of claim 19, wherein said substrate comprises a plurality of ADP glucose phosphorylase (EC 2.7.7.27) molecules and said test compounds comprise a label to permit identification of a test compound which binds to ADP glucose pyrophosphorylase (EC 2.7.7.27).

27. The method of claim 22, wherein said substrate comprises a plurality of glycogen synthase (EC 2.4.1.21) molecules and said test compounds comprise a label to permit identification of a test compound which binds to glycogen synthase (EC 2.4.1.21).

28. A compound capable of inhibiting the growth of S. mutans in a mammalian patient identified by the method according to claim 14.

29. A method of treating an S. mutant infection in a patient which comprises administering to said patient an effective amount of a compound identified by the method according to claim 14.

30. A method of treating an S. mutant infection in a patient which comprises administering to said patient a toothpaste or mouthwash comprising an effective amount of a compound identified by the method according to claim 14.

31. A method of identifying a compound capable of inhibiting the infectivity/pathogenicity of S. mutans by interfering with energy storage or utilization in said microorganism which comprises identifying a compound that inhibits the activity of glycogen synthase (EC 2.4.1.21).

32. A method of identifying a compound capable of inhibiting the infectivity/pathogenicity of S. mutans by interfering with the activity of glycogen synthase (EC 2.4.1.21) which method comprises incubating a sample of bacteria in a solution containing a known amount of ADP glucose in the presence or absence of a test compound, and assessing the effect on chain elongation of ADP glucose in the presence of said test compound, compared with the level of chain elongation in the absence of said test compound, indicates that said test compound interferes with the activity of glycogen synthase (EC 2.4.1.21).

33. A method of identifying a compound capable of inhibiting the infectivity/pathogenicity of S. mutans by interfering with the activity of glycogen synthase (EC 2.4.1.21) which method comprises exposing a substrate comprising glycogen synthase (EC 2.4.1.21) to a plurality of test compounds and identifying a test compound which binds to said glycogen synthase (EC 2.4.1.21).

34. The method of claim 33, wherein said substrate comprises a plurality of glycogen synthase (EC 2.4.1.21) molecules and said test compounds comprise a label to permit identification of a test compound which binds to glycogen synthase (EC, 2.4.1.21).

35. A compound capable of inhibiting the infectivity/pathogenicity of S. mutans in a mammalian patient identified by the method according to claim 31.

36. A method of treating an S. mutant infection in a patient which comprises administering to said patient an effective amount of a compound identified by the method according to claim 31.

37. A method of treating an S. mutant infection in a patient which comprises administering to said patient a toothpaste or mouthwash which comprises an effective amount of a compound identified by the method according to claim 31.

38. A pharmaceutical composition for the treatment of an S. mutans infection which comprises a pharmaceutically acceptable carrier and an effective antimicrobial amount of a compound identified by the method according to claim 31.

39. A pharmaceutical composition in the form of a toothpaste or mouthwash for the treatment of an S. mutans infection which comprises a pharmaceutically acceptable carrier and an effective antimicrobial amount of a compound identified by the method according to claim 31.

40. A pharmaceutical composition according to claim 10 in the form of a mouthwash or toothpaste.
